(19) United States
(12) Patent Application Publication
(10) Pub. No.: US 2006/0147916 A1
(43) Pub. Date: Jul. 6, 2006

(54) Probe carrier and method of producing same

(76) Inventors: Toru Ishibashi, Tokyo (JP); Yoshikatsu Okada, Yokohama-shi (JP); Katsuaki Kuge, Atsugi-shi (JP)

Correspondence Address: MORGAN & FINNEGAN, L.L.P., 3 WORLD FINANCIAL CENTER, NEW YORK, NY, 10281-2101, US

(21) Appl. No.: 10/521,305

(22) PCT Filed: Jul. 18, 2003
(86) PCT No.: PCT/JP03/09161

§ 371 Date: Jan. 14, 2005

(30) Foreign Application Priority Data
Jul. 19, 2002 (JP) 2002-211147
Jul. 16, 2003 (JP) 2003-197920

(51) Int. Cl. C12Q 1/68; C12M 1/34

(52) U.S. Cl. 435/6; 435/287.2

(57) ABSTRACT

A method of producing a probe carrier in which a probe is immobilized to a substrate is disclosed, which comprises providing the substrate and contacting a basic group introduced to the substrate with the probe having an acidic group to thereby immobilize the probe to the substrate. This method enables production of a probe carrier that can reduce the number of steps performed for immobilizing the probe to the substrate and easily immobilize the probe.

### TECHNICAL FIELD

The present invention relates to a high density array (DNA chip) having aligned on a solid surface a number of DNA fragments and oligonucleotides useful for simultaneous analyses of expression of genes, mutations, polymorphisms and so forth.

### BACKGROUND ART

Various methods have been known, which immobilize a probe that can specifically bind to a target substance on a substrate. Specifically, such methods include a method in which synthesis of a probe is performed on a substrate to immobilize the probe to the substrate and a method in which a preliminarily provided probe is given onto a substrate by means of a pin or stamp to immobilize the probe to the substrate.

As the immobilizing method in which synthesis of a probe is performed on a substrate, there has been known a method in which, as described in, for example, U.S. Pat. No. 5,143,8545, protective groups are removed from a selected area of a substrate by an activator and a monomer having a removable protective group is repeatedly bound to the selected area to synthesize polymers having various sequences on the substrate.

Further, as the method in which a preliminarily provided probe is given onto a substrate to immobilize the probe to the substrate, there has been known a method in which, as described in, for example, Japanese Patent Application Laid-Open No. 8-23975, a material for immobilizing a probe, composed of a substrate and a polymeric compound having a carbodiimide group carried on the substrate, is brought into contact with a biologically active substance having reactivity with a carbodiimide group to thereby immobilize the probe to the substrate. Further, as described in Japanese Patent Application Laid-Open No. 8-334509, there has been known a method in which in detecting a biologically active substance, a probe is immobilized to a compound having carbodiimide through the carbodiimide group to thereby detect the substance.

Further, in Japanese Patent Application Laid-Open No. 2001-178442, there is described a method of immobilizing a DNA fragment onto a solid phase carrier surface by contacting a DNA fragment having a thiol group at the terminal thereof with a solid phase carrier onto a surface of which a liner molecule having a reactive substituent that can form a covalent bond when it is reacted with a thiol group is immobilized at one terminal thereof to thereby form a covalent bond between the DNA fragment and the chain molecule. Specific examples of the reactive substituent that can react with a thiol group to form a covalent bond disclosed in the above-mentioned document includes substituents containing a group selected from the group consisting of a maleimidyl group, an α,β-unsaturated carbonyl group, an α-halocarbonyl group, a halogenated alkyl group, an aziridine group, and a disulfide group.

The method of immobilizing a probe to a substrate using ionic bonds includes, as described in Analytical Biochemistry 292, 250-256, for example, a method in which an aminosilane coupling agent is immobilized to a solid phase and a probe is immobilized by an interaction between the positive charge of the amino group of the aminosilane coupling agent and the negative charge of the phosphate moiety of an oligonucleotide.

However, in the method of immobilizing a probe described in Japanese Patent Application Laid-Open No. 2001-178442, immobilization of a probe to a surface of a solid phase carrier is realized by causing reaction of a probe having a thiol group on a surface of a solid phase carrier, which surface is treated by contacting a silane coupling agent, which has a substituent to be introduced onto the surface of the solid phase carrier, with the surface of the solid phase carrier to introduce the substituent and thereafter causing a substance, which has a terminal that reacts with the silane coupling agent and a terminal that reacts with a thiol group, to react with the substituent. That is, to immobilize the probe having a thiol group onto the surface of the solid phase carrier, a further treatment is required after the treatment of the surface of the solid phase carrier with the silane coupling agent.

Further, in the method in which the interaction between the positive charge of the amino group and the negative charge of the phosphate moiety of the oligonucleotide is used for immobilization of a probe, the binding state between the phosphate group and the amino group on the substrate is uncertain so that the ionic bond between the probe and the carrier is affected by ionic strength of the solution used in the hybridization reaction and subsequent washing step. This may affect the results of analysis subsequently performed.

### DISCLOSURE OF THE INVENTION

It is therefore an object of the present invention to provide a method of immobilizing a probe to a substrate in which the number of treatment steps performed for immobilizing the probe to the substrate is reduced and which can achieve simple and firm immobilization.

In order to attain the above-mentioned object, the present invention relates to a probe carrier having immobilized thereto a probe that is specifically bindable to a target substance, the probe being immobilized to the carrier through the following substances:
a) a linker bound to the probe;
b) a first functional group bound to the linker; and
c) a second functional group bound to the first functional group,

wherein a combination of the first functional group and the second functional group comprises an acidic functional group and a basic functional group.

Further, the present invention relates to a method of immobilizing the probe described above.

Further, the present invention relates to an apparatus for producing the probe carrier described above.

Further, the present invention relates to a detection method comprising imparting an analyte containing a substance to be detected to the probe carrier described above, and detecting the substance to be detected in the analyte bound to the probe carrier.

Further, the present invention relates to a detection apparatus comprising means for imparting an analyte containing a substance to be detected to the probe carrier described above, and means for detecting the substance to be detected in the analyte bound to the probe carrier.

Further, the present invention also relates to a method of selecting available combinations of functional groups by using NMR.

### BRIEF DESCRIPTION OF THE DRAWING

FIG. 1 is a graph showing results of Example 11.

### BEST MODES FOR CARRYING OUT THE INVENTION

The present invention relates to a probe carrier comprising a solid phase carrier and a probe immobilized to the solid phase carrier, the probe being capable of specifically binding to a target substance through: (a) a linker; (b) a first functional group contained in the linker; and (c) a second functional group contained in the linker, and to a method of immobilizing a probe.

While the substrate as a solid phase carrier is not particularly limited so far as it causes no harm in immobilizing a probe thereto and detecting a substance to be detected (target substance) using the obtained probe-immobilized substrate, in particular, it is advantageous that, when introducing a second functional group that is not directly bound to a linker, the substrate is treated with a silane coupling agent having the second functional group or a functional group from which the second functional group can be derived. The substrate is not particularly limited so far as the reaction with the silane coupling agent proceeds efficiently. Specifically, quarts, glass, silica, alumina, talc, clay, aluminum, aluminum hydroxide, iron, mica and the like are preferable. Oxides such as titanium oxide, zinc white, and iron oxide can also be used. Also, when detection of a target substance and versatility of the substrate as a material are taken into consideration, alkali-free glass or quartz substrate materials containing no alkali component are particularly preferable.

When a resin is used as the substrate, the affinity of the resin for the silane coupling agent must be improved, for example, by silanating the resin. For silanation, there can be used, for example, a method in which an olefin and a silane coupling agent having a vinyl group are copolymerized to produce a silanated polyolefin or a method in which a surface of a polymer having a carboxyl group is treated with a silane coupling agent having an epoxy group. However, the silanation method is not limited to the above so far as the affinity for the silane coupling agent can be improved.

Further, surface treatment of a resin having a functional group may be performed to convert the functional group into a second functional group having basicity or acidity. Alternatively, a resin having a group exhibiting basicity or acidity in a side chain of or at a terminal of the polymer may be used.

Typical examples of the functional group as a second functional group having basicity include an amino group. In the case where an amino group is selected as the second functional group, examples of the silane coupling agent having an amino group include N-β-(aminoethyl)-γ-aminopropyltrialkoxysilane, N-β-(aminoethyl)-γ-aminopropylmethyldialkoxysilane, γ-aminopropyltrialkoxysilane, γ-aminopropylmethyldialkoxysilane, N,N-dimethylaminopropyltrialkoxysilane, N-methylaminopropyltrialkoxysilane, and N-phenyl-γ-aminopropyltrialkoxysilane. It is desirable that the amino group contained in the silane coupling agent has some basicity. In particular, where a mercapto group is used for a probe, it is preferable that the amino group has a dissociation constant of 1.0×10⁻⁶ or more. Further, in consideration of the reactivity of the functional group, primary or secondary amino groups are preferable because of less steric hindrance. The alkoxysilyl group is preferably a methoxysilyl group or an ethoxysilyl group that can be hydrolyzed at high rates.

Note that in the case of substituents having the property of a Bronsted base, such as an amino group, the dissociation constant in water of such a group is given by an equilibrium constant (formula 2) of an equilibrium formula represented by formula 1.
NR₃+H₂O_{←}^{→}NHR₃⁺+OH⁻ (Formula 1)
Wherein R=H or an organic substituent such as alkyl or aryl.

$$K = \frac{[ {NH R}_{3}^{+} ] [ {OH}^{-} ]}{[ {N R}_{3} ]} \qquad ( Formula 2 )$$

In the case where such a basic silane coupling agent is used, the silane coupling agent ionically binds to a probe having an acidic functional group through a linker. Examples of the acidic functional group include a mercapto group (—SH), a sulfonato group (—SO₃⁻), and a carboxyl group (—COOH). In particular, the combination of an amino group with a mercapto group provides a relatively strong ionic bond as described in J. Colloid Interface Sci., 195 (1997) 338.

On the contrary, there is a method in which a solid phase carrier is treated with a silane coupling agent having as a second functional group an acidic group such as a carboxyl group or a mercapto group or a functional group that can be derived therefrom, and is ionically bound to a probe having a basic functional group through a linker. However, in the case of a mercapto group, it tends to be dimerized through a disulfide bond, so that it is preferable to prevent dimerization from occurring.

Further, in the case where a nucleic acid probe having a nucleic acid is used in the present invention, it is advantageous that either one of acid groups is preferably a functional group having an acidity higher than that of a phosphate group that constitutes the nucleic acid probe. This can provide a probe can be held as it is as immobilized to the solid phase even under conditions where a phosphate group and an amino group are dissociated. That is, even when drastic treatment at the time of hybridization is performed, a preferable chip that can give proper analysis results can be provided.

Note that the silane coupling agent in the present invention refers to a compound which has an organic functional group that can react with an organic compound such as a resin and has a part thereof that can bind to an inorganic compound such as glass through a siloxane bond.

The shape of the substrate is not particularly limited. However, taking a DNA chip as an example, the substrate is preferably in the form of a plate in consideration of versatility of a detection method and apparatus. Further, the plate material is preferably a plate material having a high surface smoothness, specifically, a plate of a size of 1 inch×3 inches and a thickness of about 0.7 to about 1.5 mm.

When a surface of the substrate is treated with a silane coupling agent, it is preferred to wash the surface of the substrate in advance. As the washing method, many kinds of washing methods have been known such as washing with water, washing with a solution of a chemical, washing with plasma, and washing with UV ozone. However, as a method that can simply and uniformly wash the surface of a substrate, washing with a solution of a chemical is preferred. A suitable washing method may vary depending on the kind of the substrate. For example, in the case where glass is used as a substrate, there may be mentioned of a method in which a surface of a substrate is sufficiently washed with an aqueous solution of sodium hydroxide having a predetermined concentration to remove contaminants attached on the substrate. Specifically, an aqueous solution of 1 mol/l sodium hydroxide heated to about 60° C. is provided and the surface of the substrate is wiped in the aqueous solution or brushed while the aqueous solution is showered thereon to demonstrably remove the dirt or contaminants attached on the substrate. After removing the dirt, excessive sodium hydroxide is washed out sufficiently with water. Finally, the moisture is removed by, for example, a method in which an inert gas such as N₂ is blown onto the surface of the substrate.

As the method of coating a silane coupling agent, an immersing method (a dipping method), a spin coating method, a spray coating method, a water surface casting method and so forth can be used. In particular, the immersing method that allows simple and uniform treatment is preferable. In this case, the treatment is performed preferably as follows. That is, a washed substrate is immersed in an aqueous solution of a silane coupling agent having a concentration of 0.1 to 2.0 wt % and after completion of the reaction, an excess of the solution containing the silane coupling agent is washed off. However, the concentration of the aqueous solution and the coating method are not particularly limited.

Further, it is preferable to remove the excessive silane coupling agent from the substrate and dry it by heating it at a temperature of about 100 to 120° C.

Taking the aminosilane coupling agent as an example, the basic amino group contained in the silane coupling agent immobilized to the substrate as described above accompanies formation of a hydrogen bond with a mercapto group, which is an acidic substituent introduced to the probe through a linker or protonation of the amino group so that they can interact with each other through ionic bond. This enables immobilization of the probe onto the substrate. For this reason, it is desirable that the amino group contained in the silane coupling agent has a certain degree of basicity.

The interaction with a mercapto group that is an acidic substituent introduced into the probe together with the basic amino group can be easily observed by NMR spectra. Therefore, to easily evaluate amino groups having a basicity suited for being introduced onto a surface of a substrate, NMR spectra can be used. That is, when an alkylthiol, for example, propanethiol, that imitates a probe to which a mercapto group has been introduced and an aminosilane coupling agent that is considered to be suited for immobilizing the probe or an amine compound, for example, N-propylethylenediamine, that imitates the aminosilane coupling agent are mixed in a protic solvent such as deuterium oxide and observed for NMR spectra, then remarkable low field shift is observed in chemical shift values of signals assigned to the amine compounds. On the other hand, a marked high field shift is observed in chemical shift values of signals assigned to thiol. Also, measurement of two-dimensional NMR spectra makes it possible to observe the interaction between the above-mentioned amine compound and the alkylthiol. The amino group corresponding to the amine compound exhibiting such changes is a group that has a basicity suited for immobilizing a probe to which a mercapto group has been introduced. Further, direct evaluation of the height of basicity of the amine compound used enables selection of the amino groups.

Further, the probe used in the present invention includes proteins (including complex proteins), nucleic acids, sugar chains (including glycoconjugates), lipids (including conjugated lipids), and the like biopolymers. Specifically, the probe includes enzymes, hormones, pheromones, antibodies, antigens, haptens, peptides, synthetic peptides, DNA, synthetic DNA, RNA, synthetic RNA, PNA, synthetic PNA, gangliosides, lectins, and so forth. The amount of the probe contained in a medium is as follows. That is, in the case of, for example, a nucleic acid probe, it is preferable to adjust the amount of the nucleic acid probe such that a 2mer to 500mer, particularly a 2mer to 80mer nucleic acid is contained in the medium in a concentration of 0.05 to 500 μmol/l, particularly 0.5 to 50 μmol/l.

In the case where the first functional group is introduced to a probe, it is introduced through a linker. Here, the term “linker” means a substance that exists between the probe and the first functional group and links the probe to the first functional group. The linker is not limited particularly so far as it achieves such an object. A methylene chain or a polyether chain is preferable. Further, a linear linker that has 1 to 20 atoms is preferable.

For the introduction of the first functional group to the probe, a compound having a linker is provided between a functional group that reacts with the probe and the first functional group and the compound is reacted with the probe. In this case, in order to prevent the first functional group from participating in the reaction with the probe, it is preferable to protect the first functional group with a protective group and deprotect the protective group of the first functional group after completion of the reaction with the probe.

Specifically, when for example, a mercapto group is introduced to the 5′-terminal of a DNA probe that is automatically synthesized, 5′-Thiol-Modifier C6 (manufactured by Glen Research) and the like can be used at the time of the synthesis by a DNA automated synthesizer. Further, when an amino group is introduced, 5′Amino-Modifier 5 (manufactured by Glen Research) and the like can be used at the time of the synthesis by a DNA automated synthesizer.

Note that if the first functional group including a mercapto group can be introduced efficiently, the method of introduction and the kind of linker are not particularly limited.

The combination of a basic functional group and an acidic functional group according to the present invention realizes immobilization of a probe to a solid phase through a static bond unlike a complete covalent bond. In the combination of a basic functional group and an acidic functional group according to the present invention, what is important is that the probe immobilized to the solid phase is not dissociated during the analysis. For example, in the case where a nucleic acid is immobilized to a probe and hybridization reaction with a target nucleic acid is to be detected, it is important that the probe is always stably bonded to the solid phase even under the conditions of pH, salt concentration and temperature at the time of hybridization and at the time of washing step after the hybridization. For this purpose, conventionally, a strong bond is formed between the probe and the solid phase by a covalent bond. However, as a result of studies by the inventors of the present invention, it has been found that formation of a relatively strong static bond instead of a covalent bond can achieve this object. The present invention has been made based on this finding.

Further, it is preferable that a combination of functional groups, that is, an acidic functional group having a dissociation constant of 1.0×10⁻¹² or more and a basic functional group having a dissociation constant of 1.0×10⁻⁶ or more is used.

For imparting a probe, an aqueous liquid prepared by dissolving or dispersing the probe in an aqueous medium is spotted on a substrate having a basic group by, for example, an ink jet method, a pin method or a pin and ring method.

However, the present invention is not limited to those methods but may use a method using a photolithographic technique. In addition, the spots may be of various shapes, such as circles, rectangles, and polygons. The diameter of spots is advantageously 5 μm to 500 μm.

As for a spotting method, particularly, the ink jet method is preferable since it can perform high density and accurate spotting among the above-mentioned various spotting methods. The ink jet method means a method in which a solvent containing a probe is charged in a very thin nozzle. Then, a part near a tip of the nozzle is instantaneously pressurized or heated to eject accurately an extremely small amount of the solvent containing the probe from the tip of the nozzle and allows the solvent to fly onto a surface of a substrate, thereby attaching the solvent containing the probe to the surface of the substrate.

In the spotting method by an ink jet method, the component contained in the probe medium is not particularly limited so far as the component gives substantially no influence on the probe when the component is ejected from an ink jet head in the form of a probe medium and the component has a medium composition that enables normal ejection onto a substrate by using an ink jet head. For example, in the case where the ink jet head is a bubble jet head that has a mechanism in which the ink jet head imparts thermal energy onto the medium to eject the thermal energy, a liquid containing glycerol, thiodiglycol, isopropyl alcohol, and acetylene alcohol is a preferred component contained in the probe medium. More specifically, a liquid containing 5 to 10 wt % of glycerol, 5 to 10 wt % of thiodiglycol, and 0.5 to 1 wt % of acetylene alcohol is used advantageously as a probe medium. Further, in the case where the ink jet head is a piezo jet head that ejects a medium by using a piezoelectric element, a liquid containing ethylene glycol and isopropyl alcohol is preferable as a component contained in the probe medium. More specifically, a liquid containing 5 to 10 wt % of ethylene glycol and 0.5 to 2 wt % of isopropyl alcohol is used preferably as a probe medium.

When the probe medium thus obtained is ejected through an ink jet head onto a substrate, a shape of the spot is circular and does not extend in ejected area. When the probe medium is spotted at a high density, the spots can be effectively prevented from joining between adjacent spots. Note that the characteristics of the probe medium of the present invention are not particularly limited to those described above.

Further, an aqueous liquid obtained by preliminarily dissolving or dispersing a probe and a silane coupling agent having an interactive action with an organic functional group introduced into the probe in an aqueous medium may be contacted with a surface of a substrate by a proper method such as an ink jet method or a pin method to perform introduction of the organic functional group onto the substrate and immobilization of the probe simultaneously.

Further, to lessen drying of the probe in the spot, a high boiling point substance may be added to the aqueous liquid in which the probe is dissolved or dispersed. The high boiling point substance is preferably a substance that is soluble in the aqueous liquid in which the probe is dissolved or dispersed and has not so high a viscosity. Examples of such substances include glycerol, ethylene glycol, diethylene glycol, thiodiglycol, dimethyl sulfoxide and low-molecular hydrophilic polymers. Examples of the hydrophilic polymers include polyvinyl alcohol, polyvinylpyrrolidone, paogen, carboxymethylcellulose, hydroxyethylcellulose, dextran, pullulan, polyacrylamide, polyethylene glycol, and sodium polyacrylates, etc. More preferably, ethylene glycol or diethylene glycol is used as the high boiling point substance. The concentration of the high boiling point substance is preferably in the range of 0.1 to 10 vol % in the aqueous liquid in which the probe is dissolved or dispersed. The solid phase carrier after imparting the probe may be placed in an environment at a humidity of 90% or more and a temperature range of 20 to 50° C.

After the spotting, it is preferable that excessive probe is removed by washing. Although time required for immobilization may vary depending on the kind of probe, the probe is immobilized within one minute in the case where solid phase carrier treated with an aminosilane coupling agent and a single-strand DNA probe having introduced thereto a mercapto group are used. It is preferred that the excessive probe be removed after leaving ten minutes or more.

The probe-immobilized substrate thus obtained is suitable as a probe-immobilized substrate for the detection of a target substance.

Here, with a view to increasing the precision of detection (S/N ratio) in the case where detection or the like of a target substance is performed by using the probe-immobilized substrate, for example, blocking may be performed after the immobilization of the probe onto the surface of the solid phase so that the probe-non-bound portion on the substrate does not bind to the target substance or the like contained in an analyte (sample). The blocking is performed by immersing the substrate in an aqueous solution of 0.5 to 2% bovine serum albumin for about 10 minutes to about 2 hours, for example.

However, the blocking operation may vary in optimal method and conditions depending on the kind of the second functional group. For example, in the case where an amino group is present on the surface of the substrate, the blocking method in which the above-mentioned aqueous bovine serum albumin solution is effective. In addition, a method in which the substrate is treated with acid anhydride such as acetic anhydride or succinic anhydride to cap amino groups for the purpose of preventing ionic bonding between the target and an amino group is available. However, generally, the ionic bond between a target substance, for example nucleic acid or oligonucleotide and an amino group on the substrate is a weak bond as compared with the bond according to the present invention, so that the ionic bond between the target and the substrate can be selectively removed by washing the substrate with a liquid having a strong ionic strength after the hybridization.

Note that it is sufficient for these blocking steps to be performed as needed. For example, when supply of a sample to the probe-immobilized substrate is performed to each spot limitedly and substantially no sample is attached to sites other than the spots, the blocking does not have to be performed. In the case where a sample is also attached to sites other than the spots, necessity of the blocking may vary depending on the kinds of the material that constitutes the substrate and of the second functional group. On the other hand, in the case where a substance such as a silane coupling agent having a basic group and a probe medium containing a probe having a mercapto group are spotted on a substrate that is made of glass, quartz, or the like, no blocking operation is necessary.

The probe-immobilized substrate thus prepared may be designed in various forms depending on the purpose for which they are used. For example, the probe-immobilized substrate is constructed so as to have a plurality of spots containing the same probe or a plurality of spots containing different probes. The kind, amount, and arrangement of probes may be changed properly as needed. Then the probe-immobilized substrates in which a probe or probes are arranged in a high density by various methods are used for the detection of a target substance and identification of the base sequence of a target substance and for other purposes. For example, in the case where the probe-immobilized substrate is used for detecting a single-strand nucleic acid that is a target substance whose base sequence has been already known and that is potentially contained in a sample, a known detection is performed as follows. That is, a single-strand nucleic acid having a base sequence complementary to that of the single-strand nucleic acid as a target substance is used as a probe and a probe-immobilized substrate having arranged on a solid phase a plurality of spots containing the probe is provided. To each spot on the probe-immobilized substrate is imparted a sample containing a substance to be detected and the probe-immobilized substrate is put under conditions where the single-strand nucleic acid as a target substance and the probe hybridize with each other and presence or absence of the hybrid in each spot is detected by a known method, for example, by using fluorescence, luminescence, electric current, or radioisotope but not particularly limited thereto. This enables detection of presence or absence of the target substance in the sample.

Further, in the case where the probe-immobilized substrate is used for identifying the base sequence of a single-strand nucleic acid as a target substance contained in a sample, the operation is performed as follows. That is, first, a plurality of candidate base sequences of a single-strand nucleic acid as a target substance are set and single-strand nucleic acid having respective base sequences complementary to the set base sequences are spotted on the substrate as probes. Then, a sample is supplied to each spot and the probe-immobilized substrate is put under conditions where the single-strand nucleic acids as a target substance and the probe hybridize with each other. Thereafter, presence or absence of a hybrid in each spot is detected by a known method such as fluorescence detection. This enables identification of the base sequence of a single-strand nucleic acid as a target substance. Further, other application for the probe-immobilized substrate of the present invention may include, for example, screening of a specific base sequence that a DNA-bound protein recognizes and screening of a chemical substance having the property of binding to a DNA.

It is preferable that hybridization is performed by imparting an aqueous liquid having dissolved or dispersed therein labeled sample nucleic fragments to the DNA chip prepared as described above. The hybridization is performed preferably in a temperature range of room temperature to 70° C. in a time range of 2 to 20 hours. After completion of the hybridization, the probe-immobilized substrate is washed with a mixed solution composed of a surfactant and a buffer solution to remove unreacted sample nucleic acid fragments. It is preferable to use a citrate buffer, a phosphate buffer, a borate buffer, a Tris buffer, a Good's buffer and the like as the buffer solution. It is particularly preferable to use a citrate buffer.

The feature of the hybridization using a DNA chip is to use a very small amount of a labeled sample nucleic acid fragment. For this reason, the optimal conditions for hybridization must be set depending on the chain length of the DNA fragment immobilized to the solid phase carrier and the kind of the labeled sample nucleic acid fragment. For the analysis of gene expression, it is preferable to carry out hybridization for a long period of time so that low expression genes can be sufficiently detected. For detecting a single base mismatch (single nucleotide polymorphism), it is preferable to carry out short-time hybridization. A further feature of the hybridization using a DNA chip is that comparison of expression amount or quantitative determination on the same DNA chip can be made by providing two kinds of sample nucleic acid fragments labeled with different fluorescent substances, respectively and simultaneously using the labeled sample nucleic acid fragments in hybridization.

### EXAMPLES

Hereinafter, the present invention will be described in more detail by way of examples.

### Example 1

### (1) Preparation of a Substrate

A slide glass as a glass substrate was immersed in an aqueous solution of 1 mol/l sodium hydroxide previously warmed to 60° C. for 10 minutes. Subsequently, the slide glass was sufficiently rinsed in flowing pure water to wash and remove sodium hydroxide attached to the slide glass. After sufficient rinsing, the slide glass was immersed in pure water and ultrasonic washing was performed for 10 minutes. After the ultrasonic washing, the slide glass was sufficiently rinsed in flowing pure water to wash and remove particles attached to the slide glass. Thereafter, the slide glass was dried by spin drying.

An aminosilane coupling agent (trade name: KBM-603; Shin-Etsu Chemical Co., Ltd.) was dissolved in water to a concentration of 1 wt % and the solution was stirred for 30 minutes. The slide glass was immersed in the aqueous solution for 30 minutes and then taken out and washed with water. This was placed in an oven and dried at 120° C. for 1 hour.

### (2) Synthesis of a Probe

In this example, a single-strand nucleic acid that has a base sequence that is complementary to that of all or a part of a target nucleic acid to be detected and detects the target nucleic acid by specifically hybridizing (cross-reacting) with the base sequence of the target nucleic acid was used as a probe. By using a DNA automated synthesizer, two single-strand nucleic acids, i.e., SEQ ID No:1 and SEQ ID No:2 that differs SEQ ID No:1 by only one base were synthesized. Note that at the terminals of the two single-strand DNAs, i.e., SEQ ID No:1 and SEQ ID No:2, were introduced mercapto groups by using Thiol-Modifier (manufactured by Glen Research Corporation) at the time of synthesis using the DNA automated synthesizer. Subsequently, usual deprotection was performed, DNA was recovered, the DNA was purified by high performance liquid chromatography and the obtained DNA was used in the following experiments.
5′HS-(CH₂)₆—O—PO₂—O-ACTGGCCGTCGTTTTACA3′ (SEQ ID No:1)
5′HS-(CH₂)₆—O—PO₂—O-ACTGGCCCTCGTTTTACA3′ (SEQ ID No:2)

### (3) Immobilization of the Probe

Two kinds of DNA fragments (SEQ ID No:1 and SEQ ID No:2) synthesized as described above were each dissolved in an aqueous solution containing 7.5 wt % of glycerol, 7.5 wt % of urea, 7.5 wt % of thiodiglycol, and 1 wt % of acetylene alcohol (trade name: Acetylenol E100; Kawaken Fine Chemicals Co., Ltd.) to 0.6 OD. Note that 1 OD means an amount such that oligonucleotide is dissolved in 1 ml of a medium and an absorbance of the solution at 260 nm in a cell having a light path length of 1 cm is 1.

The aqueous solutions containing DNA fragments were separately spotted on the slide glass prepared by the method described in (1) above by using a bubble jet printer (trade name: BJ-F850; manufactured by Canon, Inc., modified so as to be usable in plate printing, with the distance between the bubble jet head and the slide glass being about 1 mm and discharge amount being about 4 pl). On this occasion, observation with a 15-fold loupe indicated no satellite spot (spot derived from a droplet of a liquid spotted on a surface of the solid phase).

The slide glass on which a solution containing a probe was spotted was left to stand at room temperature for 10 minutes and then washed with 1 M NaCl/50 mM phosphate buffer (pH 7.0).

### (4) Blocking Hybridization Reaction

Bovine serum albumin was dissolved in 1 M NaCl/50 mM phosphate buffer (pH 7.0) and the DNA chip prepared by the above-mentioned method was immersed in the solution at room temperature for 2 hours to carry out blocking reaction.

A labeled DNA fragment was synthesized by connecting rhodamine to the 5′-terminal of a DNA fragment having a nucleic acid sequence complementary to that of the probe of SEQ ID No:1 and the DNA fragment was dissolved in 1 M NaCl/50 mM phosphate buffer (pH 7.0) to 50 mM. The DNA chip after the blocking treatment was immersed in the solution containing the DNA fragment and left to stand at 45° C. for 2 hours. Thereafter, unreacted DNA fragments were washed off with 1 M NaCl/50 mM phosphate buffer (pH 7.0) and the DNA chip was further washed with pure water.

### (5) Results

The DNA chip subjected to hybridization was subjected to fluoroscopic measurement at a wavelength of 532 nm by using a fluorescent scanner (trade name: Gene Pix 4000B; manufactured by Axon Instruments, Inc.). The results show that each spot was approximately circular and had a diameter of 45 μm. When measured at a PMT of 400 V and a laser power of 100%, the intensity of fluorescence attributable to the probe of SEQ ID No:1 was 21,692 and the intensity of fluorescence attributable to the probe of SEQ ID No:2, which is a single base mismatch of SEQ ID No:1 was 13,346. Further, the intensity of fluorescent of the background around the spot was 419. This clearly indicates that a single base mismatch can also be detected by the present invention.

### Example 2

### (1) Preparation of a Substrate

A substrate was prepared in the same manner as in Example 1 except that KBM-903 (trade name, manufactured by Shin-Etsu Chemical Co., Ltd.) was used as an amino silane coupling agent.

### (2) Immobilization of a Probe

The probe of SEQ ID No:1 was dissolved in an aqueous solution containing 7.5 wt % of glycerol, 7.5 wt % of urea, 7.5 wt % of thiodiglycol, and 1 wt % of acetylene alcohol (trade name: Acetylenol E100; Kawaken Fine Chemicals Co., Ltd.) to 0.6 OD. This was spotted on the same slide glass in the same manner as in Example 1.

### (3) Blocking Hybridization Reaction

Blocking hybridization reaction was performed in the same manner as in Example 1.

### (4) Results

Each spot was approximately circular and had a diameter of 45 μm. When measured at a PMT of 400 V and a laser power of 100%, the intensity of fluorescence was 29,998 and the intensity of fluorescence of the background around the spot was 393.

### Example 3

A DNA chip was prepared in the same manner as in Example 2 except that KBM-602 (trade name, manufactured by Shin-Etsu Chemical Co., Ltd.) was used as an amino silane coupling agent, and then a blocking reaction and a hybridization reaction were performed. After these reactions, fluorescence was observed.

The results show that each spot was approximately circular and had a diameter of 40 μm. When measured at a PMT of 400 V and a laser power of 100%, the intensity of fluorescence attributable to the probe of SEQ ID No:1 was 20,675. Further, the intensity of fluorescent of the background around the spot was 442.

### Example 4

A DNA chip was prepared in the same manner as in Example 2 except that N-methyl aminopropyl trimethoxy silane (manufactured by CHISSO CORPORATION) was used as an amino silane coupling agent, and then a blocking reaction and a hybridization reaction were performed. After these reactions, fluorescence was observed.

The results show that each spot was approximately circular and had a diameter of 50 μm. When measured at a PMT of 400 V and a laser power of 100%, the intensity of fluorescence was 22,246. Further, the intensity of fluorescent of the background around the spot was 212.

### Example 5

### (1) Preparation of a Substrate

A slide glass as a glass substrate was immersed in an aqueous solution of 1 mol/l sodium hydroxide previously warmed to 60° C. for 10 minutes. Subsequently, the slide glass was sufficiently rinsed in flowing pure water to wash and remove sodium hydroxide attached to the slide glass. After sufficient rinsing, the slide glass was immersed in pure water and ultrasonic washing was performed for 10 minutes. After the ultrasonic washing, the slide glass was sufficiently rinsed in flowing pure water to wash and remove particles attached to the slide glass. After that, the slide glass is dried thorough spin drying.

### (2) Immobilization of the Probe

An aminosilane coupling agent KBM-603 (trade name, manufactured by Shin-Etsu Chemical Co., Ltd.) was dissolved in an aqueous solution containing 7.5 wt % of glycerol, 7.5 wt % of urea, 7.5 wt % of thiodiglycol, and 1 wt % of acetylene alcohol (trade name: Acetylenol E100; Kawaken Fine Chemicals Co., Ltd.) and stirred for 20 minutes. Subsequently, the synthesized DNA fragment (SEQ ID No:1) was dissolved in the solution containing the silane coupling agent to 0.6 OD.

The solution containing the silane coupling agent and the DNA fragment was spotted on the slide glass prepared by the method described in (1) above by using a bubble jet printer (trade name: BJ-F850; manufactured by Canon, Inc., modified so as to be usable in plate printing). On this occasion, observation with a 15-fold loupe indicated no satellite spot (spot derived from a droplet of a liquid spotted on a surface of the solid phase).

The slide glass on which a solution containing the silane coupling agent and a probe was spotted was left to stand at room temperature for 20 minutes and then washed with 1 M NaCl/50 mM phosphate buffer (pH 7.0).

### (3) Hybridization Reaction

A labeled DNA fragment was synthesized by connecting rhodamine to the 5′-terminal of a DNA fragment having a nucleic acid sequence complementary to that of the probe of SEQ ID No:1 and the DNA fragment was dissolved in 1 M NaCl/50 mM phosphate buffer (pH 7.0) to 50 mM. The DNA chip was immersed in the solution containing the labeled DNA fragment and left to stand at 45° C. for 2 hours. Thereafter, unreacted DNA fragments were washed off with 1 M NaCl/50 mM phosphate buffer (pH 7.0) and the DNA chip was further washed with pure water.

### (4) Results

The DNA chip subjected to hybridization was subjected to fluoroscopic measurement at a wavelength of 532 nm by using a fluorescent scanner (trade name: Gene Pix 4000B; manufactured by Axon Instruments, Inc.). The results show that each spot was approximately circular and had a diameter of 45 μm. When measured at a PMT of 400 V and a laser power of 100%, the intensity of fluorescence was 4831. Further, the intensity of fluorescent of the background around the spot was 84.

This indicates that when the silane coupling agent for introducing the second functional group and the probe were imparted to the substrate simultaneously, a blocking operation is unnecessary.

### Example 6

### (1) Preparation of a Substrate

A-189 (trade name, manufactured by Nippon Unicar, Co. Ltd.) as a mercaptosilane coupling agent was dissolved in an aqueous hydrochloric acid solution at pH 4 to 0.1 wt % and stirred for 5 hours. The aqueous solution was spin-coated on a slide glass washed by the same method as described in Example 1. The slide glass was dried in an oven at 110° C. for 30 minutes.

### (2) Immobilization of the Probe

The probe of SEQ ID No:3 modified with an amino group was dissolved in an aqueous solution containing 7.5 wt % of glycerol, 7.5 wt % of thiodiglycol, and 0.01 wt % of acetylene alcohol (trade name: Acetylenol E100; Kawaken Fine Chemicals Co., Ltd.) to 0.6 OD. Spotting was performed in the same manner as in Example 1.
5′H₂N—(CH₂)₆—O—PO₂—O-ACTGGCCGTCGTTTTACA3′ (SEQ ID No:3)

### (3) Blocking Hybridization Reaction

Blocking hybridization reaction was performed in the same manner as in Example 1.

### (4) Results

The results show that each spot was approximately circular and had a diameter of 30 μm. When measured at a PMT of 700 V and a laser power of 100%, the intensity of fluorescence was 1700. Further, the intensity of fluorescent of the background around the spot was 63.

### Example 7

### (1) Preparation of a Substrate

A substrate was prepared in the same manner as in Example 5.

### (2) Immobilization of the Probe

A mercaptosilane coupling agent (trade name: A-189; manufactured by Nippon Unicar, Co. Ltd.) was dissolved to 0.1 wt % in a solution prepared by adding hydrochloric acid to an aqueous solution containing 7.5 wt % of glycerol, 7.5 wt % of thiodiglycol, and 1 wt % of acetylene alcohol (trade name: Acetylenol E100; Kawaken Fine Chemicals Co., Ltd.) at pH 4, and was stirred for 1 hour. Subsequently, the synthesized DNA fragment (SEQ ID No:3) was dissolved in the solution containing the silane coupling agent to 0.6 OD. Subsequently, the synthesized DNA fragment (SEQ ID No: 3) was dissolved in the solution containing the silane coupling agent to 0.6 OD.

The solution containing the silane coupling agent and the DNA fragment was spotted on the slide glass prepared by the method described in (1) above by using a bubble jet printer (trade name: BJ-F850; manufactured by Canon, Inc., modified so as to be usable in plate printing). On this occasion, observation with a 15-fold loupe indicated no satellite spot (spot derived from a droplet of a liquid spotted on a surface of the solid phase).

The slide glass on which a solution containing the silane coupling agent the probe was spotted was left to stand at room temperature for 20 minutes and then in an oven at 80° C. for 30 minutes and washed with 1 M NaCl/50 mM phosphate buffer solution (pH 7.0).

### (3) Hybridization Reaction

A hybridization reaction was performed in the same manner as in Example 5.

### (4) Results

The DNA chip subjected to hybridization was subjected to fluoroscopic measurement at a wavelength of 532 nm by using a fluorescent scanner (trade name: Gene Pix 4000B; manufactured by Axon Instruments, Inc.). The results show that each spot was approximately circular. When measured at a PMT of 400 V and a laser power of 100%, the intensity of fluorescence was 4527. Further, the intensity of fluorescent of the background around the spot was 32.

### Example 8

Presence or absence of the interaction between the amino group contained in the aminosilane coupling agent and the mercapto group of the probe was observed by NMR spectra using an alkylthiol that imitates the mercapto group-introduced probe (1-propanethiol) and an amine compound, N-propylethylenediamine, that imitates the amino group moiety in KBM-603 and KBM-602.

### (1) Preparation of a Sample

A solution of N-propylethylenediamine (manufactured by Across Corp.) in D₂O (manufactured by Aldrich) solution (600 μl) was charged in a 5-mmφ NMR tube, to which was added 1-propanethiol (manufactured by Tokyo Kasei Kogyo Co., Ltd.) in a molar ratio of about 1:1 with respect to N-propylethylenediamine and its change was observed by ¹H NMR.

### (2) Results

Hereinbelow, respective chemical shift values of N-propylethylenediamine, 1-propanethiol, and those after mixing both are shown.

¹H NMR Spectrum Data (400 MHz, D₂O, Room Temperature)

N-Propylethylenediamine

δ/ppm

0.79 (methyl-H of a propyl group)

1.37 (methylene-H at the β-position of a propyl group)

2.42 (methylene-H at the α-position of a propyl group)

2.51 (methylene-H at the β-position of an NH₂ group)

2.61 (methylene-H at the α-position of an NH₂ group)

1-propanethiol

δ/ppm

0.87 (methyl-H of a propyl group)

1.53 (methylene-H at the β-position of a propyl group)

2.45 (methylene-H adjacent to an SH group)

N-propylethylenediamine after mixing each

δ/ppm

0.81 (methyl-H of a propyl group)

1.43 (methylene-H at the β-position of a propyl group)

2.54 (methylene-H at the α-position of a propyl group)

2.63 to 2.66 (methylene-H at the β-position of an NH₂ group)

2.68 to 2.71 (methylene-H at the α-position of an NH₂ group)

1-propanethiol after mixing each

δ/ppm

0.82 (methyl-H of a propyl group)

1.42 (methylene-H at the β-position of a propyl group)

2.33 (methylene-H adjacent to an SH group)

As a result, by mixing both model compounds, amine and thiol, a change was observed in which the chemical shift of the signal assigned to N-propylethylenediamine underwent a low field shift while the chemical shift of the signal assigned to 1-propanethiol underwent a high field shift. In particular, signals near the amino group and mercapto group shifted in greater amounts and a change in coupling was observed for the signal near the amino group.

### Example 9

Presence or absence of the interaction between the amino group in the aminosilane coupling agent and the mercapto group of the probe was observed by NMR spectra using an alkylthiol that imitates the mercapto group-introduced probe (1-propanethiol) and an amine compound, propylamine, that imitates the amino group moiety in KBM-903.

### (1) Preparation of a Sample

A solution of propylamine (manufactured by Tokyo Kasei Kogyo Co., Ltd.) in D₂O (manufactured by Aldrich) (600 μl) was charged in a 5-mmφ NMR tube, to which was added 1-propanethiol (manufactured by Tokyo Kasei Kogyo Co., Ltd.) in a molar ratio of about 1:1 with respect to propylamine and its change was observed by ¹H NMR spectra.

### (2) Results

Hereinbelow, respective chemical shift values of propylamine, 1-propanethiol, and those after mixing both are shown.

¹H NMR Spectrum Data (400 MHz, D₂O, Room Temperature)

propylamine

δ/ppm

0.81 (methyl-H of a propyl group)

1.37 (methylene-H at the β-position of a propyl group)

2.51 (methylene-H at the α-position of a propyl group)

1-propanethiol

δ/ppm

0.87 (methyl-H of a propyl group)

1.53 (methylene-H at the β-position of a propyl group)

2.45 (methylene-H adjacent to an SH group)

propylamine after mixing each

δ/ppm

0.86 (methyl-H of a propyl group)

1.50 (methylene-H at the β-position of a propyl group)

2.72 (methylene-H at the α-position of a propyl group)

1-propanethiol after mixing each

δ/ppm

0.84 (methyl-H of a propyl group)

1.45 (methyl-H at the β-position of a propyl group)

2.36 (methylene-H adjacent to an SH group)

As a result, by mixing both model compounds, amine and thiol, a change was observed in which the chemical shift of the signal assigned to propylamine underwent a low field shift while the chemical shift of the signal assigned to 1-propanethiol underwent a high field shift. In particular, signals near the amino group and mercapto group shifted in greater amounts.

### Example 10

Presence or absence of the interaction between the amino group in the aminosilane coupling agent and the mercapto group of the probe was observed by NMR spectra using an alkylthiol that imitates the mercapto group-introduced probe (1-propanethiol) and an amine compound, N-methylpropylamine, that imitates the amino group moiety in N-methylaminopropyltrimethoxysilane.

### (1) Preparation of a Sample

A solution of N-methylpropylamine (manufactured by Aldrich) in D₂O (manufactured by Aldrich) solution (600 μl) was charged in a 5-mmφ NMR tube, to which was added 1-propanethiol (manufactured by Tokyo Kasei Kogyo Co., Ltd.) in a molar ratio of about 1:1 with respect to N-methylpropylamine and its change was observed by ¹H NMR spectra.

### (2) Results

Hereinbelow, respective chemical shift values of N-methylpropylamine, 1-propanethiol, and those after adding 1-propanethiol to N-methylpropylamine are shown.

¹H NMR Spectrum Data (400 MHz, D₂O, Room Temperature)

N-methylpropylamine

δ/ppm

0.81 (methyl-H of a propyl group)

1.40 (methylene-H at the β-position of a propyl group)

2.23 (methyl group)

2.43 (methylene-H at the α-position of a propyl group)

1-propanethiol

δ/ppm

0.87 (methyl-H of a propyl group)

1.53 (methyl-H at the β-position of a propyl group)

2.45 (methylene-H adjacent to an SH group)

N-methylpropylamine after mixing each

δ/ppm

0.87 (methyl-H of a propyl group)

1.54 (methylene-H at the β-position of a propyl group)

2.49 (methyl group)

2.75 (methylene-H at the α-position a propyl group)

1-propanethiol after mixing each

δ/ppm

0.84 (methyl-H of a propyl group)

1.44 (methyl-H at the β-position of a propyl group)

2.35 (methylene-H adjacent to an SH group)

As a result, by mixing both model compounds, amine and thiol, a change was observed in which the signal assigned to N-methylpropylamine underwent a low field shift while the signal assigned to 1-propanethiol underwent to a high field shift. In particular, signals near the amino group and mercapto group shifted in greater amounts.

### Example 11

A slide glass was washed in the same manner as in Example 1 and N-methylaminopropyltrimethoxysilane (manufactured by Chisso Corporation) was dissolved in water to 0.3 wt % the mixture was stirred for 20 minutes. The slide glass was immersed in the obtained aqueous solution for 20 minutes and then taken out and washed with water, dried in an oven at 120° C. for 1 hour. On the slide glass was spotted the probe of SEQ ID No:1 in the same as in Example 2.

Sodium chloride was dissolved in 10-mM phosphate buffer so as to obtain dilutions having a sodium chloride concentration of 0, 100, 300, 500, or 1,000 mM and the prepared DNA chip was washed with these solutions. The washing method was as follows. First, the DNA chip was irradiated with an ultrasonic wave for 2 minutes in these solutions, rinsed with the same solution, and stirred overnight in the same solution.

The washed DNA chip was subjected to blocking and hybridization reaction in the same manner as in Example 2 and then fluorescence observation was performed.

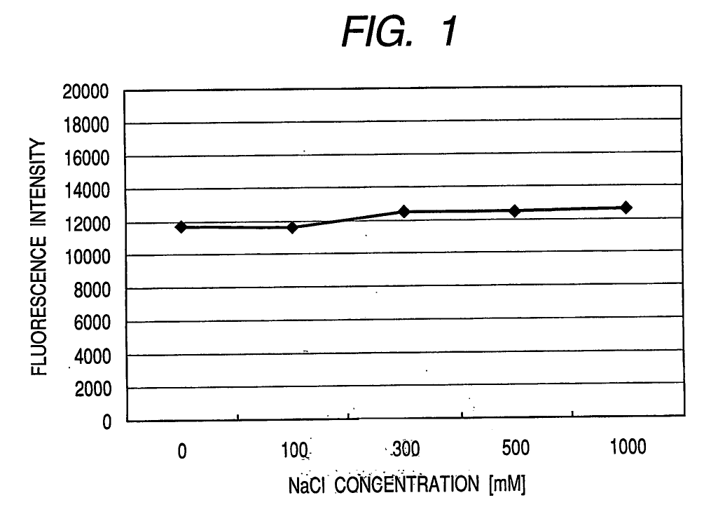

As shown in FIG. 1, it was demonstrated that in spite of changes in the concentration of a salt, the intensity of fluorescence was not influenced so that the probe can be stably immobilized through ionic bonds.

### INDUSTRIAL APPLICABILITY

According to the present invention, a probe carrier is provided in which a probe can be immobilized to a substrate by a simple method and is stable even when an ionic strength is changed.

Further, a DNA chip that can detect even a single base mismatch can be manufactured by using a nucleic acid probe as a probe.

1. A probe carrier having immobilized thereto a probe that is specifically bindable to a target substance, the probe being immobilized to the carrier through the following substances:
a) a linker bound to the probe;
b) a first functional group bound to the linker; and
c) a second functional group bound to the first functional group,
wherein a combination of the first functional group and the second functional group comprises an acidic functional group and a basic functional group.

2. The probe carrier according to claim 1, wherein the combination of the first functional group and the second functional group comprises an acidic functional group having a dissociation constant of 1.0×10⁻¹² or more and a basic functional group having a dissociation constant of 1.0×10⁻⁶ or more.

3. The probe carrier according to claim 1, wherein the probe comprises an oligonucleotide or a nucleic acid.

4. The probe carrier according to claim 3, wherein the oligonucleotide or the nucleic acid has the linker at a 3′-terminal or a 5′-terminal thereof.

5. The probe carrier according to claim 1, wherein the linker comprises a methylene chain or a polyether chain.

6. The probe carrier according to claim 1, wherein the acidic functional group is a mercapto group and the basic functional group is an amino group.

7. The probe carrier according to claim 1, wherein the basic functional group is one selected from the group consisting of a primary amino group, a secondary amino group, and a mixture thereof.

8. The probe carrier according to claim 1, wherein the probe has a second functional group introduced by treatment of the solid phase carrier with a silane coupling agent.

9. The probe carrier according to claim 8, wherein the solid phase carrier is one selected from the group consisting of glass, quarts, silica, and a mixture thereof.

10. The probe carrier according to claim 1, wherein the combination of the first functional group and the second functional group is a combination that causes shift of mutual chemical shifts of signals in the NMR spectrum by binding each other.

11. A detection method comprising the steps of:
imparting an analyte containing a substance to be detected to a probe carrier according to claim 1; and
detecting the substance to be detected in the analyte bound to the probe carrier.

12. A detection apparatus using a detection method according to claim 11.

13. An apparatus for producing a probe carrier according to claim 1.

14. A method of immobilizing a probe that is specifically bindable to a target substance to a solid phase carrier, comprising the steps of:
providing a probe having a linker containing a first functional group;
providing an immobilization substrate having a second functional group;
imparting the probe to the immobilization substrate; and
binding the first functional group and the second functional group to each other,
wherein a combination of the first functional group and the second functional group comprises an acidic functional group and a basic functional group.

15. The method of immobilizing a probe according to claim 14, wherein the combination of the first functional group and the second functional group comprises an acidic functional group having a dissociation constant of 1.0×10⁻¹² or more and a basic functional group having a dissociation constant of 1.0×10⁻⁶ or more.

16. The method of immobilizing a probe according to claim 14, wherein the probe comprises an oligonucleotide or a nucleic acid.

17. The method of immobilizing a probe according to claim 16, wherein the oligonucleotide or the nucleic acid has the linker at a 3′-terminal or a 5′-terminal thereof.

18. The method of immobilizing a probe according to claim 14, wherein the linker comprises a methylene chain or a polyether chain.

19. The method of immobilizing a probe according to claim 14, wherein the acidic functional group is a mercapto group and the basic functional group is an amino group.

20. The method of immobilizing a probe according to claim 14, wherein the basic functional group is one selected from the group consisting of a primary amino group, a secondary amino group, and a mixture thereof.

21. The method of immobilizing a probe according to claim 14, wherein the probe has a second functional group introduced by treatment of the solid phase carrier with a silane coupling agent.

22. The method of immobilizing a probe according to claim 21, wherein the solid phase carrier comprises one selected from the group consisting of glass, quarts, silica, and a mixture thereof.


SEQUENCE LISTING (3 sequences)

SEQ ID NO: 1 - DNA, 18 bp, ARTIFICIAL
  probe
actggccgtcattttaca

SEQ ID NO: 2 - DNA, 18 bp, Artificial
  probe
actggccctcgttttaca

SEQ ID NO: 3 - DNA, 18 bp, Artificial
  probe
actggccatcgttttaca